(19) United States
(12) Patent Application Publication
(10) Pub. No.: US 2006/0246546 A1
(43) Pub. Date: Nov. 2, 2006

(54) Manufacture of biologic cellular products

(76) Inventors: Lauri Laurene Jenkins, Newark, DE (US); Jay Irving Kennedy, Circleville, OH (US); Victor Sisto Lusvardi, Chadds Ford, PA (US); Colleen Merritt Severyn, Media, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/412,858

(22) Filed: Apr. 27, 2006

Related U.S. Application Data
(60) Provisional application No. 60/676,336, filed on Apr. 29, 2005.

(51) Int. Cl. C12P 21/06; C12N 9/22; C12M 3/00

(52) U.S. Cl. 435/69.1; 435/199; 435/289.1

(57) ABSTRACT

The manufacture of cellular product such as a protein by expression from a cell line in a bioreactor is carried out wherein the bioreactor is a container made of flexible film, at least the interior surface of the container being fluoropolymer, and in addition, the nutrient medium and other agents used in the fermentation or cell culture process can be prepared in a separate container of the same film.

### BACKGROUND OF THE INVENTION

1. Field of the Invention

This invention relates to vessels used in the manufacture of biologic cellular products.

2. Description of Related Art

Protein therapies are made by culturing of cells taken from a cell line. Typically, the cell line is recombinant, i.e. one or more cells are genetically altered by combination with DNA from a different organism, and these recombinant cells are cloned to form a cell bank of the cell line. Aliquots are taken from this cell bank for culturing, and the therapeutic protein is expressed during growth (propagation) of the cells in the cell culture process. In the case of the cell line being recombinant, the resultant expressed protein is also recombinant. The expression of the protein is typically carried out by inoculating a fermentation broth (bacterial cell line) or cell culture medium (mammalian cell line) with the aliquot of the cell line into a nutrient medium into which is bubbled oxygen and nitrogen and accompanied by mixing so that the fermentation or cell culturing conditions within the medium is homologous and controlled. The vessel in which this expression is carried is called a bioreactor. Typically, the reaction is carried out in a succession of at least two bioreactors in increasing volume, this succession being called an inoculum train, the increase in volume designed to establish the best condition for expression of the protein therapy within each bioreactor and thus in the overall manufacturing process. The current favorite material of construction of the bioreactor is stainless steel, thought to be corrosion resistant to the reaction medium, but found to be somewhat deficient in this regard as indicated by the need for periodic shut-down of the bioreactor, clean out, electropolishing of the inner reactor surface, sterilizing the inner surface and validation of the “refurbished” vessel for suitability before being placed back into service. These stainless steel vessels for commercial operation are typically at least 10 liters in volume. Similar vessels are used to prepare the nutrient medium for feeding into the bioreactor(s). The disadvantages of the stainless steel vessels are their high initial cost and high operating costs arising from the need for periodic shutdowns, as well as the loss of therapeutic protein caused by contamination coming from the stainless steel vessel. An additional disadvantage is the problem of cross-contamination, i.e. batch-to-batch contamination in the bioreactor(s).

The inadequacy of stainless steel vessels in the process for expressing therapeutic proteins extends to other processes for producing biologic cellular products, i.e. proteins in general, including non-therapeutic proteins that would be used in recombinant processes, and non-proteins such as toxins and polysaccharides. This inadequacy also extends to the stainless steel vessels (perfusion reactor) used for extractions from the cell culture, principally of toxins and polysaccharides.

There are several patents that disclose the use of polymer bags for fermentation, but the production of cellular products such as for human therapy, recombinant technology, toxins or polysaccharides is not disclosed. In this regard, U.S. Pat. No. 5,565,015 discloses the use of a small bag of polypropylene as a fermenter to make mushroom spores. U.S. Pat. No. 6,432,698 discloses a 1 to 200 liter disposable bioreactor bag made of PVC for the fermentation to produce a biological pesticide. The PVC bag of '015 would be especially disadvantageous in the manufacture of the above-mentioned cellular products, because of the presence of phthalate plasticizer in PVC that would be a source of contamination of such products.

U.S. Pat. No. 4,847,462 discloses the laser manufacture of disposable bags made from films of various fluoropolymers, for use for culturing (growing) cells taken from the human body for eventual return to the human body, thereby providing cellular immunotherapy. The laser seaming operation is carried out in a box that is kept sterile and which includes germicidal lamps to prevent mold growth and kill all bacteria. The film is water-rinsed as an additional measure for achieving film cleanliness. An air knife directing sterile air to dry the water rinsed film is also disclosed.

The problem remains on how to provide a non-contaminating bioreactor, which also avoids the problem of cross contamination.

### BRIEF SUMMARY OF THE INVENTION

The present invention solves this problem by providing a disposable non-contaminating bioreactor. Thus, in the process of expressing a cellular product from a cell line, the improvement comprises carrying out said process in a container made of flexible film, at least the interior surface of said film being fluoropolymer. The fluoropolymer film container that forms the vessel that is the bioreactor is non-contaminating, and upon completion of the expression (fermentation or cell culture) process and emptying of the bioreactor, the film container can be discarded, and a new film container is used, within which the next batch of fermentation or cell culture and accompanying expression of the cellular product is carried out. This eliminates cross-contamination. The shut-down between batches of the cell culture/expression process is limited to discarding the old film container and installing a new film container as the new bioreactor. There is no clean-out or electropolishing. The requirement for validation of the sterilized condition of the interior surface of the film container is preferably avoided by having the new film container be sterilized prior to use as the new bioreactor. The process improvement described above can be used as each of the bioreactors in an inoculum train.

The cell culturing occurring in the expression of a different cellular product in accordance with the present invention differs from the cell culturing that involves only the growing of the original cells, without creating a different cellular product. The latter cell culturing is represented for example by the immunotherapy application for the disposable bags in U.S. Pat. No. 4,847,462. In contrast, the cell culturing occurring in the expression process of the present invention produces a non-living cellular product from the living, growing cell culture. This different cellular product is more susceptible to harm from organics contamination than the host cell culture. The host cell culture is a living organism and can therefore make some adjustment to counteract such contamination. The expressed cellular product, because it is not a living organism, cannot make this adjustment. Consequently, the likelihood of an organic (contamination) to organic (cellular product) reaction to adversely affect the cellular product is much greater. In addition, the cellular product is a small fraction of the cell culture from which the cellular product is expressed. Therefore, an amount of organics contamination that might be small relative to the cell culture, will be large relative to the amount of cellular product. Another difference from mere cell culturing is that the expression process is usually carried out in a succession of reactors of increasing volume, within each of which the cell culturing is conducted until optimum density is reached. The cell culture/expressed cellular product medium is thus exposed to a plurality of bioreactor surfaces in advancing from one bioreactor to the next, thereby being subject to contamination by each reactor surface, instead of exposure to only one container surface as in the case of mere cell culturing, i.e. unaccompanied by expression of different product. The surprising resistance to extraction of organics from the fluoropolymer forming at least the interior surface of the container(s) (bioreactors) in which the expression process of the present invention is carried out enables the cellular product to be preserved as formed for supply to the step(s) of recovering this product from the biomass within which it was produced. This is especially surprising when the container is sterilized prior to use as the bioreactor by exposure to degradative ionizing radiation, such as by gamma radiation, as will be further described hereinafter.

In a preferred embodiment, the nutrient medium in which the expression process is carried out is prepared in a separate container made of the same flexible film. After preparation of the nutrient medium, portions thereof are transferred to the film container within which the protein expression process is carried out. This embodiment can be carried out in combination with the expression process describe above or independent thereof.

In another preferred embodiment, one or more other agents that might be used in the expression process in the bioreactor can also be prepared in separate containers made of flexible film, at least the interior surface of the film being fluoropolymer. Such other agents include activator (induction agent), buffer, acid and base. The storage of the nutrient medium and other agents will typically be carried out separately in the same flexible film container within which the nutrient medium or other agent was prepared.

In another preferred embodiment, the cloning of the cell line to form the cell bank is carried out in a separate container of the same flexible film. This embodiment can be carried out in combination with either the expression process or the nutrient medium and/or other agent preparation process or independent thereof.

By carrying out the nutrient medium and other agent preparation/storage and bioreaction in containers of the same flexible film, the expressed cellular product and materials used to produce such product all see the same container material surface, which eliminates the variability in cellular product manufacture that can accompany the use of different surfaces in the manufacturing process. The same is true when this commonality extends back to the cloning process.

### DETAILED DESCRIPTION OF THE PRESENT INVENTION

The cellular products that can be made by expression from a cell line include toxins, polysaccharides, and proteins. The proteins include peptides and can be therapeutic or non-therapeutic. Examples of therapeutic proteins include recombinant vaccines, enzymes of therapeutic value such as TPA, antigens and antibodies. Examples of non-therapeutic proteins include the enzymes used for recombinant technology, i.e. cloning, such enzymes including RNAse, DNAse, Ligase, and restriction endonucleases. The term “expression” as used herein also includes intracellular product, i.e. expressed cellular product contained within the cell, which can be obtained by lysing the cell culture to recover the desired expressed cellular product. The cell culture/expression process in which the cellular product, whether naturally occurring or recombinant, is expressed typically involves the mixing of the aliquot of the cell bank (inoculum) into a nutrient medium and possibly additional purified water (WFI described hereinafter) and other agent as described above, under controlled temperature and with bubbling of oxygen and nitrogen through the nutrient medium, all to produce a homologous bioreaction medium, so that reaction conditions in one region of the nutrient medium are as close the same as in all other regions in the medium. The same is true for transfer of fermentation broth or cell culture medium from one bioreactor to another in an inoculum train, wherein optimum conditions are maintained in each bioreactor for production of the desired cellular product. When the optimum amount of cell growth (cell density) is obtained in one bioreactor, the fermentation broth or cell culture is transferred to a larger volume bioreactor, wherein optimum conditions are established to increase the production of the cellular product. In each bioreactor, the nitrogen purges carbon dioxide from the nutrient medium, which is formed during the expression process, and optimum amounts of nutrient medium, oxygen and carbon dioxide are maintained to provide optimum cell growth and cellular product production. The optimum pH is also established, monitored and maintained. The nutrient medium is typically an aqueous medium and the fermenting cells or the culturing cells are preferably dispersed to form a suspension within the nutrient medium. These conditions are well known in the art and are individualized for the particular cell line being fermented or cultured and the particular cellular product being formed.

The preparation of nutrient media is also well known in the art and is individualized as just described. The function of the nutrient medium to make the cells of the particular cell line in the bioreactor grow and in the course of growing, express the desired cellular product, such as a protein. The nutrient medium is an aqueous solution typically including an energy source, usually one or more sugars, to stimulate cell growth, and will typically include additional ingredients, such as minerals, amino acids, and vitamins, to mimic natural biologic fluid stimulative of cell growth for the particular cell line. The nutrient medium will also include strong acid(s) or base(s) and buffers to define and control pH of the nutrient medium, and some or all of these ingredients and others in the nutrient medium corrode or otherwise extract contaminants from the stainless steel bioreactor surface in contact with the medium, both in the preparation and fermentation and cell culturing processes. The acids, bases, and/or buffers in the nutrient medium are highly corrosive reagents and can come from the preparation and storage of these other agents in the flexible film containers as described above. In addition, these other agents can be added to the bioreactor independent of the nutrient medium as needed. Examples of ingredients in the nutrient medium include calcium chloride solution, glucose, lactalbumin hydrolysate, soy hydrolysate, glutamine, sodium pyruvate, and tryptose phosphate broth. Nutrient media are sometimes purchased from nutrient medium manufacturers and are sometimes prepared by the protein manufacturer, by mixing the nutrient medium ingredients with water in a container. In either case, this container can be used for preparation and storage of the nutrient medium, or separate containers can be used. Typically the nutrient medium is pumped through a sterilizing filter (microorganism size exclusion filter) into the bioreactor(s) for carrying out the expression process.

The cloning of a cell produced by recombinant DNA is also well known in the art and likewise requires freedom from contamination to obtain the desired result.

The present invention provides a container (vessel) for the preparation and/or storage of the nutrient medium, other agents, and for the bioreaction in which the cellular product is expressed within the nutrient medium, such container providing much improved resistance to contaminating the nutrient medium and thus the therapeutic protein, and as described above, improved economy and of operation. The present invention also provides the container for extraction of expressed cellular product, such as toxin or polysaccharide from the cell culture from which these entities are expressed. Typically, such extraction is carried out in a perfusion reactor, and the container used in the present invention can be this reactor. The toxin after extraction separation is then treated such as by reaction with formaldehyde, to be converted to a therapeutic toxoid.

The container used in the present invention as the vessel for carrying out the nutrient medium and other agent preparation and storage and the fermentation or cell culture bioreaction, the extraction of toxin or polysaccharide from microorganism, and even the cloning process is made of flexible film, the surface of which forming the interior surface of the container is fluoropolymer.

The fluoropolymers used in the present invention are melt-fabricable, which means that they are sufficiently flowable in the molten state (heated above its melting temperature) that they can be fabricated by melt processing, preferably by extrusion such as to form a film that is optically clear. Typically, the fluoropolymer by itself is melt-fabricable; in the case of polyvinyl fluoride, the fluoropolymer is mixed with solvent for extrusion, i.e. solvent-aided extrusion. The resultant film has sufficient strength so as to be useful. The melt flowability of the fluoropolymer can be described in terms of melt flow rate as measured in accordance with ASTM D-1238, and the fluoropolymers of the present invention preferably have a melt flow rate of at least 1 g/10 min, determined at the temperature which is standard for the particular fluoropolymer; see for example, ASTM D 2116a and ASTM D 3159-91a. Polytetrafluoroethylene (PTFE) is generally not melt processible, i.e. it does not flow at temperatures above the melting temperatures, whereby this polymer is not melt-fabricable. PTFE film is also not optically clear. Optical clarity is desired so that when the film is fabricated into a container, the interior of the container can be observed through the film wall of the container, enabling the observer to confirm that no visible contaminant or evidence of contamination such as the appearance of turbidity is present. Low molecular weight PTFE is available, called PTFE micropowder, the molecular weight being low enough that this polymer is flowable when molten, but because of the low molecular weight, the resultant molded article has no strength. The absence of strength is indicated by the brittleness of the article. If a film can be formed from the micropowder, it fractures upon flexing. In contrast, the melt-fabricable fluoropolymers used in the present invention can be formed into films that can be repeatedly flexed without fracture. This flexibility can be further characterized by an MIT flex life of at least 500 cycles, preferably at least 1000 cycles, and more preferably at least 2000 cycles, measured on 8 mil (0.2 mm) thick compression molded films that are quenched in cold water, using the standard MIT folding endurance tester described in ASTM D-2176F. The flexibility of the container enables it to collapse into a flattened shape. Flexibility can also be confirmed by attempting to puncture the film from which the container is made, such as by following the procedure of ASTM F1342, with the result that prior to puncture, the stylus used in the puncture test deflects the film from its planar disposition in the test to the extent of at least about 5 times the thickness of the film being tested, and preferably at least 10 times the film thickness.

The preferred melt-fabricable fluoropolymers for use in the present invention comprise one or more repeat units selected from the group consisting of —CF₂—CF₂—, —CF₂—CF(CF₃)—, —CF₂—CH₂—, —CH₂—CHF— and —CH₂—CH₂—, these repeat units and combinations thereof being selected with the proviso that said fluoropolymer contains at least 35 wt % fluorine, preferably at least 50 wt % fluorine. Thus, although hydrocarbon units may be present in the carbon atom chain forming the polymer, there are sufficient fluorine-substituted carbon atoms in the polymer chain to provide the desired minimum amount of fluorine present, so that fluoropolymer exhibits chemical inertness. The fluoropolymer preferably also has a melting temperature of at least 150° C., preferably at least 200° C., and more preferably at least 240° C.

Examples of perfluoropolymers, i.e., wherein the monovalent atoms bonded to carbon atoms making up the polymer are all fluorine, except for the possibility of other atoms being present in end groups of the polymer chain, include copolymers of tetrafluoroethylene (TFE) with one or more perfluoroolefins having 3 to 8 carbon atoms, preferably hexafluoropropylene (HFP). The TFE/HFP copolymer can contain additional copolymerized perfluoromonomer such as perfluoro(alkyl vinyl ether), wherein the alkyl group contains 1 to 5 carbon atoms. Preferred such alkyl groups are perfluoro(methyl vinyl ether), perfluoro(ethyl vinyl ether) and perfluoro(propyl vinyl ether). Typically, the HFP content of the copolymer is about 7 to 17 wt %, more typically about 9 to 17 wt % (calculation: HFPI×3.2), and the additional comonomer when present constitutes about 0.2 to 3 wt %, based on the total weight of the copolymer. The TFE/HFP copolymers with and without additional copolymerized monomer is commonly known as FEP. Examples of hydrocarbon/fluorocarbon polymers (hereinafter “hydrofluoropolymers”) include vinylidene fluoride polymers (homopolymers and copolymers), typically called PVDF, copolymers of ethylene (E) with TFE, typically containing 40 to 60 mol % of each monomer, to total 100 mol %, and preferably containing additional copolymerized monomer such as perfluoroalkyl ethylene, preferably perfluorobutyl ethylene. These copolymers are commonly called ETFE. While ETFE is primarily composed of ethylene and tetrafluoroethylene repeat units making up the polymer chain, it is typical that additional units of from a different fluorinated monomer will also be present to provide the melt, appearance, and/or physical properties, such as to avoid high temperature brittleness, desired for the copolymer. Examples of additional monomers include perfluoroalkyl ethylene, such as perfluorobutyl ethylene, perfluoro(ethyl or propyl vinyl ether), hexafluoroisobutylene, and CH₂═CFR_{f} wherein R_{f} is C₂-C₁₀ fluoroalkyl, such as CH₂═CFC₅F₁₀H, hexafluoropropylene, and vinylidene fluoride. Typically, the additional monomer will be present in 0.1 to 10 mol % based on the total mols of tetrafluoroethylene and ethylene. Such copolymers are further described in U.S. Pat. Nos. 3,624,250, 4,123,602, 4,513,129, and 4,677,175. Additional hydrofluoropolymers include EFEP and the copolymer of TFE/HFP and vinylidene fluoride, commonly called THV. Films of these copolymers are all commercially available. Typically the film from which the container is made will have a thickness of about 2 to 10 mils (0.05 to 0.25 mm).

The fluoropolymer forms at least the inner surface of the container, i.e. the container may be formed from a film that is laminate in which the fluoropolymer layer faces the interior of the container. Preferably however, the fluoropolymer forms the entire thickness of the film. In either case, the bag will have a film thickness as stated in the preceding paragraph. The mono (single) layer film has the advantage of avoiding the need to laminate or otherwise bond the fluoropolymer layer of the laminate to the outer layer thereof. This has the further advantage in forming seams in the fabrication of the film into a container. The seam will involve heat bonding fluoropolymer to itself and edges of the film present in the seam in the interior of the container will be entirely fluoropolymer. The fluoropolymer layer or monolayer film as the case may be is non-adherent with respect to the nutrient medium, fermentation broth and cell culture medium and the expressed cellular product, i.e. the ingredients in these media do not adhere to the fluoropolymer surface in contact with these media. The film, whether a laminate or monolayer is preferably optically clear, so that the interior of the container made from the film can be observed through the film wall of the container, enabling the observer to confirm that no visible contaminant is present when the container is supplied in a package as will be explained hereinafter.

The container can have any configuration and size desired for application in the particular step in the manufacture of cellular product. For example, the container can be formed from two sheets of film heat sealed together along their edges to form an envelope. Alternatively, the container can be formed from sheets of film to form a container with distinct bottom and sides, either to form a round-sided container or one with distinct sides coming together at corners. Whatever the configuration, the container forms a vessel, within which a step in the manufacture (expression) of cellular product can be carried out. The container can be open at the top (in use) or can be closed, except for a port of entry for the medium to be made or used. The port of entry can simply be a length of tubing heat sealed to the film forming the container. The entry port can be located elsewhere in the container and additional openings can be provided, such as equipped with tubing heat sealed to the film of the container, for such processing activities as discharge of the liquid contents from the container, feed of gas to the container, or multiple gases as in the case when the container is used as the bioreactor, wherein both oxygen and nitrogen are introduced to the fermentation broth/nutrient medium or cell culture/nutrient medium in the bag, and additional ports are provided for introduction of other agents and to enable carbon dioxide to vent from the container. An additional port can be provided for the introduction of a mixing blade into the interior of the container. Examples of bag configurations include those shown in U.S. Pat. Nos. 5,941,635, 6,071,005, 6,287,284, 6,432,698, 6,494,613, 6,453,683, and 6,684,646.

The interior volume of the container can be such as to accommodate either the research manufacture of the cellular product or the commercial manufacture thereof. Typically, the volume of the container will be at least 500 ml, but more typically, at least 1 liter, but sizes (volumes) of at least 10 liter, at least 50 liter, at least 100 liters, and at least 1000 liters, and even at least 10,000 liters are possible. Since the fluoropolymer film can be made in practically unlimited length, it is only necessary to cut this length into the lengths desired and fabricate these lengths together to form the container with the configuration and size desired. Small container sizes can be used unsupported, while a rigid support can be used for larger container sizes. The rigid support could be simply a base upon which the container rests or a rigid housing within which the bag is positioned so that both the bottom and side(s) of the container are supported. When the rigid support will be necessary will depend on the size of the container and its film thickness. The rigid support can be existing vessels used in the manufacture of cellular product, whereby the container made of flexible film forms flexible disposable liner for the vessel. The disposable liner is formed separately from the rigid support and therefore can be placed on or into the rigid support for carrying out the process of the present invention, and can be removed from the support upon completion of the process. This is in contrast to a permanent liner that is formed on and adhered to the inner surface of the vessel.

The container can be formed by heat sealing one or more sheets of film of the fluoropolymer together, depending on the size and configuration of the container. Heat sealing involves welding overlapping lengths of the film together by applying heat to the overlap. The welding is achieved by heating the overlapped surfaces, usually under pressure, such as by using a heated bar or hot air, impulse, induction, infrared laser or ultrasonic heating. The overlapping film surfaces are heated above the melting temperature of the fluoropolymer to obtain a fusion bond of the overlapping film surfaces. An example of heat sealing of overlapping films of FEP (melting temperature of about 260° C.) is as follows: A pair of hot bars are heated to 290° C. and pressed against overlapping FEP film having a total film thickness of 5 mils (0.125 mm) under a pressure for 30 psi to provide the fusion seal in 0.5 sec. Lower temperatures can be used for lower melting fluoropolymers. For ETFE overlapping films, each 4 mils (0.1 mm) thick, the hot bars of the impulse sealer are heated to 230° C. under a pressure of 60 psi (42 MPa) for about 10 sec to obtain the fusion seal. Typically the heat sealing can be completed in no more than 15 sec. Additional information on heat sealing is provided in S. Ebnesajjad, Fluoroplastics, Vol. 2, Melt Processible Fluoropolymers, published by Plastics Design Library 2003, pp. 493-496. The ports of entry into and exit from the container can be welded to the film by heat sealing techniques or by the welding and sealing techniques applied to various fluoropolymers as disclosed on pp. 461-493 of Fluoroplastics.

After fabrication of the container, because of the flexibility of the film from which it is made, the container, which is also flexible, can be collapsed as if it were a bag. The film, preferably after fabrication into a container, can be sterilized by known means, such as exposure to superheated steam or dry hot air or such chemical treatment as hot hydrogen peroxide or ethylene oxide or radiation. Ionizing radiation is preferred and gamma or electron beam (e-beam) radiation is especially preferred because of the sterilization effectiveness of radiation and its avoidance of residual chemicals from the chemical treatment sterilization of the film (container) so as not to contaminate the manufacture of the protein with such chemical or its residue. Preferably, the bag is inserted into a sealable overwrap, which is sized to enable the bag to fit within the overwrap. Alternatively, the bag may be folded over upon itself, which enables a smaller size overwrap to be used. The overwrap itself is preferably flexible and therefore formed from a polymer film such as of about 1 to 10 mils (0.025 to 0.25 mm) in thickness. Since the overwrap is not used in the manufacture of the cellular product, it does not have to have the non-contaminating character of the fluoropolymer bag with respect to the manufacture of the cellular product. Inexpensive polymer films such as of polyolefin such as polyethylene or polypropylene, or polyester, such as polyethylene terephthalate can be used as the overwrap. The polymer film making up the overwrap can be formed into a bag of the size and shape desired by heat sealing using conditions suitable for the particular polymer being used. The same heat sealing can be used to seal the overwrap once the fluoropolymer bag is inserted into the overwrap.

Sterilization can then be advantageously carried out on the package resulting from the sealed overwrap containing the fluoropolymer bag, preferably by exposing the package to ionizing radiation, preferably gamma or e-beam radiation, in an effective dosage to achieve sterilization of the fluoropolymer bag. Typically, such dosage is in the range of 25 to 40 kGy. AAMITIR 17-1997 discloses guidance for the qualification of polymeric materials that are sterilized by radiation, including certain fluoropolymers. By way of example, a bag made of two sheets of FEP film, each 5 mil (0.125 mm) thick, heat sealed together as described above on three sides to leave an open top and having a capacity of 5 liters is formed. Alternatively, the bag is made from two sheets of ETFE film, each 4 mils (0.1 mm) thick, heat sealed as described above. A bag of similar size of polyethylene terephthalate (PET) film 1.2 mil (0.03 mm) thick is also formed, and the FEP or ETFE bag is placed within the polyethylene terephthalate bag. The polyethylene terephthalate bag is heat sealed using an AudionVac-VMS 103 vacuum sealing machine operating on program 2 to heat seal the overlapping films of the PET bag with a 2.5 sec dwell time of a hot bar pressing the films together against an anvil. The machine first inflates the PET bag, followed by drawing a vacuum of 1 Bar on the interior of the bag, and then carrying out the heat sealing. The resultant vacuum sealed PET bag with the collapsed FEP or ETFE bag inside forms a flat package. The resultant package is exposed to gamma radiation from a C⁶⁰ source to provide a dosage of 26 kGy, which is a sufficient dosage to sterilize the FEP or ETFE bag within the PET overwrap. The PET overwrap maintains the sterilized condition of the FEP or ETFE bag until the PET overwrap is unsealed to make the bag available as a container for use in the process of the present invention. Terminal sterilization can also be carried out by exposing the package to steam.

A gusseted container is made by heat sealing flexible films of fluoropolymer such as FEP or ETFE together at their edges. This container when filled with liquid medium has a rectangular shape when viewed from one direction and an upstanding elliptical shape when viewed in the perpendicular direction. Thus, the container when filled (expanded) has the shape of a pillow. This container can also be oriented in the horizontal direction so that a gusseted sidewall faces upward. The orientation of the container will determine where the ports (openings) are positioned. In the embodiment next described, the container is oriented vertically, so that the gusseted sidewalls are vertical. The gussets can be formed from separate pieces of film or can be formed integrally with the sidewall. For example, a heat-sealed film in the shape of a tube can be pinched to form inwardly extended pleats, which are heat sealed at their top and bottom to retain the pleat shape, when the container is collapsed. The bottom and top of the tube shape is heat sealed to form the container. When the container is expanded, the pleats unfold at their midsections, to form gussets in the side of the container. In a different embodiment, the elliptical-shaped gusset sidewalls of the container are made from FEP or ETFE film cut into this elliptical shape. The sidewall are heat sealed to the rectangular front and back walls of the container by impulse heating, which involves a controlled heat-up applied to overlapping film portions, clamped between a heat bar and an anvil, heat sealing of the clamped film portions together, and controlled cooling of the seal while still under clamping pressure. The heat bar and anvil are shaped to the configuration needed for the desired shape of the heat seal. Three ports are provided at the top of the container spaced along the upper rectangular edge, one for ingredient addition into the interior of the container, on for venting gas, notably carbon dioxide that develops during the bioreaction, and the third port providing entry for a mixing blade. Three ports are also provided at the bottom rectangular edge of the container, one for drainage of liquid contents of the container, and the other two for introduction of oxygen and nitrogen into the interior of the container. Except for the presence of the ports, the container is a closed vessel. Each port is formed from tubing that has a valve for opening and closing the tubing. The tubing is heat sealed by impulse heating to the film walls of the container, i.e. the tubing is sandwiched between films forming opposite sides of the container and sealed around and to the periphery of the tubing. Alternatively, the ports can be integral with a base having tapered ends and the base is heat sealed to the opposing films. The interior volume of this container is 200 liters. When inflated by the addition of liquid medium, the container can be supported within a rectangular tank, the bottom edge of the container resting on the bottom of the tank, which has an aperture through which the tubing of the three bottom ports can extend, and the elliptical sidewalls being supported by the corresponding sidewalls of the tank, and the rectangular sidewalls contacting the corresponding sidewalls of the tank to provide support. After fabrication of this container, the flexibility of the FEP or ETFE film enables the container to collapse into a flat shape, which can be heat sealed into an overwrap and then sterilized by exposure of the resultant sealed package to gamma radiation as described in the preceding paragraph. The gamma radiation also sterilizes the ports heat sealed into the container.

Details of the testing for extraction of organics from polymer film containers that had been subjected to 40 kGy gamma radiation are as follows:

The container of flexible film is filled with 250 ml of water for injection (WFI) or other test liquid and the resultant filled container is heated at 40° C. for 63 days. WFI is defined in United States Pharmacopeia (USP) 1231 under Water for Pharmaceutical Purposes. In substance, WFI is highly purified water, the purity of which is designed to prevent microbial contamination and the formation of microbial endotoxins. WFI is also well known as being highly corrosive material, which provides a severe test of extractability of organics (organic compounds) from any polymer container. During the heating at 40° C. for 63 days, the highly corrosive WFI or other test solution has the opportunity to extract organics (organic compounds) from the film from which the container is made. Whether this extraction occurs or the extent of its occurrence is determined by subjecting samples of the WFI or other test liquid, as the case may be, to separation by gas chromatography, followed by analysis of the separation products by detection means. Volatile organic compounds (VOC) extracted in this process and separated in an HP 6890 GC (column: SPB-1 sulfur, 30 m×0.32 mm ID, 4.0 micrometer thick film, operating at a range of 50-180° C.) are determined using a flame ionization detector (FID). The sample of test liquid is injected into the column at a temperature at 270° C. The flame detection pattern is electronically compared to a library of patterns in order to identify the organic present in the WFI or other test liquid. The separation of individual VOCs is based on retention time in the column, and the identification of the VOCs is done by their ionization signature.

Higher molecular weight organics that might be extracted from the stored, heated container can be considered as semi-volatile organic compounds (semi-VOC) and are also subjected to separation in a GC column, followed by detection of any semi VOC present. The column used for separating samples of the WFI or other test liquid is a GC (HP 6890) column, 30 m×250 micrometer ID, utilizing a 0.25 micrometer HP-5MS film, and the separated sample passing through the column is analyzed by Mass Spectrometer (MS) analysis using an HP 5973 MSD analyzer. The sample is injected into the column at 220° C. For the semi-VOC analysis the sample of WFI or other test liquid is spiked with 1000 ppb of 2-fluorobiphenyl (internal detection standard) and extracted several times with methylene chloride. The VOCs and semi-VOCs form a boiling point continuum of the organics that might be extracted from the container of polymer film being tested. The limits of detection of the VOCs and semi-VOCs are 50 ppb. The reporting of zero (0) for detection of extractables from the WFI and other test liquids in Tables 1 and 2 means that if extractables were present, they were present in less than 50 ppb.

The heated storage conditions for the WFI or other test liquid in the flexible film container, together with the GC separation of any organics present in a sample of the test liquid after such storage in the container, and analysis of the GC effluent can simply be referred to herein as the extraction test (long term).

As stated above, with respect to the containers of flexible film of fluoropolymers and containing WFI, no organics were detected in the extraction test.

The results of subjecting bags of fluoropolymer film and of film of different polymers to WFI and to other extractants to the extraction test are shown in Tables 1 and 2.

TABLE 1
Comparison of Extraction Test Results - Semi-VOC

| | Organics in Extraction Liquid | | | |
|---|---|---|---|---|
| | After Extraction (ppb) | | | |
| Extractant liquid | FEP | ETFE | EVA* | PE** |
| WFI | 0 | 0 | 0 | 570 |
| 1N HCl, 15 wt % NaCl | 0 | 0 | 0 | 0 |
| 1N NaOH, 15 wt % NaCl | 0 | 0 | 74 | 70 |
| PBS (phosphate buffered saline) | 0 | 0 | 0 | 210 |
| Guanidine HCl | 0 | 0 | 0 | 395 |

*laminate in which ethylene/vinyl acetate copolymer is the interior layer
**laminate in which ultra low density polyethylene is the interior layer

TABLE 2
Comparison of Extraction Test Results - VOC

| | Organics in Extraction Liquid | | | |
|---|---|---|---|---|
| | After Extraction (ppb) | | | |
| Extractant liquid | FEP | ETFE | EVA | PE |
| WFI | 0 | 0 | 1395 | 2946 |
| 1 N HCl, 15 wt % NaCl | 0 | 0 | 2820 | 2961 |
| 1 N NaOH, 15 wt % NaCl | 0 | 0 | 1247 | 1997 |
| PBS (phosphate buffered saline) | 0 | 0 | 1271 | 1820 |
| Guanidine HCl | 0 | 0 | 1127 | 1200 |

These extractants (challenge solutions) shown in Tables 1 and 2 mimic liquids that may be included in the biological material by virtue of the process for producing the biologic material, such as in the manufacture of cellular product such as therapeutic protein. As shown in these Tables, the bags made of either FEP film or ETFE film were far superior to the bags made from the indicated hydrocarbon polymers as the interior layer of the bag, i.e. the hydrocarbon contact layers were much more contaminating of the various extraction test liquids. The organics detected in the extraction liquids in the EVA and/or PE bags included the following: ethanol, isopropanol, and dimethyl benzenedicarboxylic acid ester. It is surprising that the FEP and ETFE films do not yield extractables, because the effect of the gamma radiation on these polymers is to cause degradation, by polymer chain scission, this effect being more severe for the FEP than the ETFE as shown by the physical test results in Tables 3 and 4. The degradation/crosslinking effect of gamma radiation on various fluoropolymers is discussed in Y. Rosenberg et al., “Low Dose Υ Irradiation of Some Fluoropolymers; Effect of Polymer Structure”, J. Applied Science, 45, John Wiley & Sons, 783-795.

The variability of the extraction results with the hydrocarbon polymer bags, i.e. different challenge liquids give different extraction results for the same bag, is a cause of concern for the user because the extraction results with still different reagents, as may be encountered in use, are unpredictable. In contrast, the consistently low extraction values for the fluoropolymers gives confidence that this will extend to different reagents.

Another test was conducted in which samples of the bags mentioned above were exposed to desorption conditions in a clean stainless steel tube of a Perkin-Elmer ADT-400. The tube was heated at 50° C. for 30 min to generate volatiles from the bag sample. The resultant gases were then subjected to GC separation (HP 6890 GC) at a column temperature of 40° C. to 280° C. and calibrated with n-decane, and mass spectrometer analysis (HP 5973 MS detector). This is the outgas test. The detection limit is 1 ppm (1 microgram/gm). No outgas was detected for either the FEP film or the ETFE film. For the PE film, 67 ppm of organics were detected, which included isopropyl alcohol, branched alkane hydrocarbons, octane, alkene hydrocarbons, decane, dodecane, alkyl benzenes, 2,6-di-tert-butyl benzoquinone, 1,4-benzenedicarboxylic acid, dimethyl ester, and 2,4-bis(1,1-dimethylethyl)-phenol. For the EVA film, 140 ppm of organics were detected, which included acetic acid, heptane, octane, branch alkane hydrocarbon, octamethyl cyclotetrasiloxane, decamethyl cyclopentasiloxane, alkyl benzenepolysiloxane, alkyl phenol, and 2,6-di-tert-butyl benzoquinone.

The container made from film of either tetrafluoroethylene/hexafluoropropylene copolymer or ethylene/tetrafluoroethylene copolymer exhibits far superior stability under exposure to conditions of extraction and outgasing.

The effect of gamma radiation on physical properties of several fluoropolymers was tested. Tensile strength and elongation was tested on extruded films 4 to 5 mil (102-127 micrometers) thick in accordance with ASTM D 638, before and after exposure to 40 KGy gamma radiation, with the results being as reported in Table 3.

TABLE 3
Tensile Strength and Elongation of Fluoropolymer films

| | ETFE | PVDF | FEP |
|---|---|---|---|
| Tensile strength, psi (MPa) | | | |
| Before radiation | (62.3)8900 | (56)8000 | (42.7)6100 |
| After radiation | (52.5)7500 | (56.7)8100 | (26.6)3800 |
| Elongation at break, % | | | |
| Before radiation | 430 | 310 | 460 |
| After radiation | 440 | 140 | 450 |

These results show that the radiation greatly weakens the PVDF (polyvinylidenefluoride) and FEP (tetrafluoroethylene/hexafluoropropylene copolymer), greatly reduced tensile strength in the case of FEP and greatly reduced elongation in the case of PVDF. The reduction in elongation for PVDF manifests itself as reduced flexibility for the film making up the container, making it prone to cracking upon flexing.

The effect of 40 kGy of gamma radiation on fluoroethylene (polytetrafluoroethylene) is even more severe than for the PVDF and FEP. Both tensile strength and elongation deteriorate to lower levels than for the PVDF and FEP.

The films forming the subject of testing, the results of which are shown in Table 3,were also subjected to tear resistance testing in accordance with ASTM D 1004-94a, wherein the test specimen has a notch stamped therein as shown in Fig. 1 of the ASTM test procedure. In this test, the test specimen is gripped between pairs of jaws and pulled apart at a rate of 51 mm/min, which concentrates the stress at the notch in the test specimen. As the jaws are pulled apart, a graph of load required vs. extension of the test specimen in the notch region is formed. The resultant curve is plotted until the load reaches a peak and then declines 25% from the peak or until the specimen breaks, whichever occurs first. The area under the curve as determined by the computer program MathCAD represents the energy required to break the film. This test simulates the localized stresses that might be imposed on the container made from the film, such as might be encountered by contact with a sharp object or development of internal pressure within the liquid contents of the container. High load accompanied by low elongation in the tear resistance test has the disadvantage that the film will tend to puncture rather than elongate when subjected to localized stress. Moderate load accompanied by high elongation provide greater resistance to puncture. Table 4 shows the energy to break for the films of Table 3.

TABLE 4
Energy to Break

| | | Energy at Break |
|---|---|---|
| Film | Gamma Radiation Dosaqe(KGy) | (cm · N/cm) |
| ETFE | 0 | 2250 |
| | 25 | 2695 |
| | 40 | 2694 |
| PVDF | 0 | 1205 |
| | 25 | 1033 |
| | 40 | 753 |
| FEP | 0 | 1085 |
| | 25 | 1819 |
| | 40 | 1567 |

These results were obtained at room temperature (15-20° C.) tear resistance testing, averaging 5 test films/radiation condition. The energy at break values are normalized to the thickness of the film being tested, which accounts for the “cm” in the denominator.

It is preferred in the present invention that the energy at break of the film after exposure to 40 kGy of gamma radiation is at least 90% of that of the film prior to the radiation exposure, more preferably is at least as great after the radiation exposure as before. Table 4 shows no loss in energy at break for the ETFE film, when exposed to gamma radiation and substantially greater energy at break than either the PVDF or the FEP.

These physical testing results show that the ethylene/tetrafluoroethylene copolymer film bag is preferred over bags made from either PVDF or FEP because of the gamma radiation sterilizability of the ethylene/tetrafluoroethylene copolymer bag without appreciable detriment to either extraction of volatile compounds or in physical properties significant to the utility of the bag. Thus, the FEP and PVDF films used to make the flexible container according to the present invention should preferably be sterilized by methods other than gamma radiation, e.g. by exposure to e-beam radiation or by exposure to steam. If gamma radiation were used to sterilize perfluoropolymer such as FEP or radiation-degraded hydrofluoropolymer such as PVDF, these fluoropolymers would preferably be in the bag to be sterilized as the interior surface (film) of a laminate, in which the outer layer(s) of the laminate would be essentially not degraded by the radiation. Examples outer layer polymers are those disclosed above for use as the overwrap of the terminally sterilized package.

1. In the process of expressing a cellular product from a cell line, the improvement comprising carrying out said process in a container made of flexible film, at least the interior surface of said container being fluoropolymer.

2. In the process of claim 1 wherein said container made of flexible film is in the form of a bag and said bag is sterilized prior to carrying out said process.

3. In the process of claim 1 wherein said container is disposable upon completion of said process.

4. In the process of claim 1 wherein said process is carried out in a succession of said containers, each made of said film, and being of increasing volume.

5. In the process of claim 1 wherein said process is carried out homologously in the presence of stirring, oxygen, and nitrogen.

6. In the process of claim 1 wherein said container is supported.

7. In the process of claim 1 wherein said fluoropolymer is hydrofluoropolymer or perfluoropolymer.

8. In the process of claim 1 wherein said film is about 2 to 10 mils thick.

9. In the process of claim 1 wherein the entire film is made of said fluoropolymer.

10. In the process of claim 1 wherein said container has a capacity of at least 500 ml.

11. In the process of claim 1, wherein said cellular product is a toxin, polysaccacharide or protein.

12. In the process of claim 11 wherein said protein is therapeutic or non-therapeutic.

13. In the process of claim 12 wherein said therapeutic protein is a recombinant vaccine, enzyme of therapeutic value, antigen, or antibody

14. In the process of claim 12 wherein said non-therapeutic protein is an enzyme useful in cloning, such as RNase, DNase, Ligase, and restriction endonuclease.

15. In the process of claim 1 wherein said process of expressing said cellular product is carried out in the presence of nutrient medium and the additional improvement of preparing said nutrient medium in a separate container made of said flexible film.

16. In the process of claim 1 wherein said process of expressing said cellular product is carried out in the presence of at least one of activator, buffer, acid or base, and the additional improvement of preparing and/or storing at least one of said activator, buffer, acid, and base in separate containers made of flexible film, at least the interior surface of each said containers being fluoropolymer.

17. In the process of claim 15 and the additional improvement of cloning the cell line that expresses said cellular product in a separate container made of said flexible film, at least the interior surface of said container being fluoropolymer.

18. In the process of claim 1 wherein said container is provided as a package containing said container in a sealed overwrap and removing said container from said overwrap so as to be available for said process of expressing said protein.

19. In the process of preparing and/or storing nutrient medium, activator, buffer, acid or base for the expression of cellular product from a cell line, the improvement comprising carrying out said preparing and/or storing of at least one of said nutrient medium, activator, buffer, acid or base in a container made of flexible film, at least the interior surface of said film being fluoropolymer.

20. In the process of claim 19 wherein said storing is carried out in said container.

21. In the process of extracting expressed cellular product from a cell culture, the improvement carrying out said extracting in a container made of flexible film, at least the interior surface of said film being fluoropolymer.
